(19) United States
(12) Patent Application Publication
(10) Pub. No.: US 2016/0278770 A1
(43) Pub. Date: Sep. 29, 2016

(54) SUTURE DELIVERY DEVICE, AND SUTURE, FOR FACILITATING FIBROSIS AND HEALING

(71) Applicant: Central Park Diagnostics, Inc., Bayonne, NJ (US)

(72) Inventor: Shahnoz Rustamova, Bayonne, NJ (US)

(21) Appl. No.: 15/082,579

(22) Filed: Mar. 28, 2016

Related U.S. Application Data
(60) Provisional application No. 62/138,576, filed on Mar. 26, 2015.

(51) Int. Cl. A61B 17/06

(52) U.S. Cl. CPC A61B 17/06166; A61B 2017/0464

(57) ABSTRACT

Various embodiments of a future delivery device and of a suture are provided. The suture delivery device, in one embodiment, comprises a sleeve over a suture and a suture wire. In one embodiment, a suture comprises a primary structure and at least one secondary structure configured to facilitate fibrosis and/or tissue healing. In one embodiment, a suture comprises one or more types of nanoparticles.

### CROSS-REFERENCE TO RELATED APPLICATIONS

The present application claims the benefit of priority of U.S. Provisional Patent Application No. 62/138,576 filed Mar. 26, 2015, entitled “SUTURE DELIVERY DEVICE AND SUTURE FACILITATING FIBROSIS,” which is incorporated by reference in the present application.

### FIELD OF THE INVENTION

Embodiments described in this disclosure relate generally to sutures and related devices, and more particularly to a suture delivery device

### BACKGROUND

Thread lifting is a known option for facial rejuvenation. It can be applied as an alternative to a face-lift, or as a means to postpone the need for one. The material used for thread lifting typically consists of thin, absorbable sutures, or non-absorbable monofilament sutures. These typically are provisioned with small bi-directional barbed threads from the same material (e.g., APTOS threads; the Quill™ tissue closure device by Surgical Specialties Corporation; or the V-Loc™ barbed wound closure device by Covidien) or with small cylinders from polylactic acid (e.g., Silhouette Sutures® by Silhouette Lift Inc.). The teeth on one end of prior art threads anchor themselves to the underlying subcutaneous tissues, and the barbs on the other end grab and gently lift the skin in the desired direction. Thus, there is a resulting gathering of facial tissue and skin, which solidifies after several days through the natural connective tissue (fibrosis). Some types of sutures are designed to approximate the tissue.

Typically, the suture delivery device used for the types of sutures described above are regular surgical needles of various shapes, forms (e.g., cog, mono, mono screw, tornado, tornado screw), and gauges. This type of delivery system for a suture requires practice and experience. Before mastering the technique, there is the potential for a physician to make mistakes at the entry point of the needle. The resulting loss of the suture is not economical. In addition, the fixation point could be difficult, reducing the efficacy of the procedure, which may result in patient dissatisfaction with the procedure. Also, sutures that are approved currently by the Food and Drug Administration (FDA) for use in the U.S. are non-absorbable—the suture remains in the tissue permanently. In case of undesirable results, removal of the suture could be difficult and, in some cases, even impossible.

### BRIEF DESCRIPTION OF THE DRAWINGS

An understanding of embodiments described in this disclosure and many of the related advantages may be readily obtained by reference to the following detailed description when considered with the accompanying drawings, of which:

FIG. 1 is a diagram of a suture with suture delivery device according to an embodiment of the present invention;

FIG. 2 is a diagram of a suture with suture delivery device according to an embodiment of the present invention;

FIG. 3 is a diagram of a suture and suture wire according to an embodiment of the present invention;

FIG. 4A, FIG. 4B, and FIG. 4C depict an example use of a suture delivery device to insert a coiled suture in tissue according to an embodiment of the present invention; and

FIG. 5A, FIG. 5B, and FIG. 5C depict an example use of a suture delivery device to insert a coiled suture in tissue according to an embodiment of the present invention.

### DETAILED DESCRIPTION

In accordance with one or more embodiments, a suture delivery device comprises a sleeve that covers a coiled/spring type of suture. The sleeve prevents the suture from uncoiling, prevents suture waste, and facilitates precise distribution to the indicated location.

Some embodiments provide for a suture facilitating fibrosis. In one embodiment, the suture is absorbable. In one embodiment, the suture comprises at least one fixating hook, such as at one end or tip of the suture, or proximate the end or tip of the suture. When the suture is employed, the hook preferably should be fixed (in anchor-like fashion) to a less mobile area of the body.

According to some embodiments, the suture may comprise one or more cogs, spikes, beads, barbs, filaments, cones, or other type of secondary structure attached to or protruding from a main, central, or primary thread of the suture. Unlike known sutures, however, in accordance with some embodiments of the present invention, the cogs, spike, barbs, filaments, cones, or beads are more flexible and are not necessarily designed to approximate tissue. Accordingly, in some embodiments, the cogs, spikes, beads, filaments, barbs, cones, or other type of secondary structure may not be used to approximate tissue. Instead, the design of the cogs, spikes, beads, filaments, barbs, or cones is configured to create a tissue stimulating effect in order to create fibrosis. Some examples of the fibrotic effect of the secondary structures are described in this disclosure with respect to the accompanying figures.

The inventive suture and suture delivery device provides advantages over prior art delivering devices and sutures because it simplifies the lifting procedure, making less room for error, while improving patient safety and satisfaction.

According to one embodiment of a suture delivery device configured to deliver non-spiral or non-coiled sutures, the suture goes through a long, straight needle with a suture wire inside the needle. This wire is configured like a fork (comprising a y-shape and/or two or more tines) to hold a tip of the suture where the anchor is located. Advantageously, this allows anchor placement to be more precise and more effective.

In some embodiments, the suture material may be coated with a substance, such as lactic acid, to create a greater fibrotic effect, and thus a greater lifting and/or rejuvenating effect on the body, improving the look and patient satisfaction.

In some embodiments, the suture may comprise a targeted agent delivery system configured to deliver one or more types of drugs, medicines, minerals, vitamins, other types of supplements, and/or plasma (e.g., collectively referred to as “agents” in this disclosure).

The targeted agent included in the delivery system may be selected, for example, to promote collagen formation (e.g., zinc (Zn), copper (Cu), vitamin C, vitamin A, vitamin D, vitamin B), provide an antibacterial effect, and/or promote regeneration of tissue (e.g., plasma and/or zinc to promote healing of organ tissue). Some examples of agents that may be useful for accelerating and/or promoting healing at the site of a wound or surgical incision include, but are not limited to: biocidal agents, antimicrobial agents, antibiotics, anti-proliferatives, medicants, growth factors, anti-clotting agents, plasma, clotting agents, analgesics, anesthetics, anti-inflammatory agents, wound repair agents and the like, chemotherapeutics, biologics, protein therapeutics, monoclonal or polyclonal antibodies, DNA, RNA, peptides, polysaccharides, lectins, lipids, probiotics, diagnostic agents, angiogenics, anti-angiogenic drugs, polymeric drugs, and combinations thereof.

In one or more embodiments, the targeted delivery system may comprise one or more nanostructures, such as a nanoparticle (NP) or carbon nanotube (CNT). Various types of NP structures and/or coatings are known to those skilled in the art. In some embodiments, the NPs may be loaded with one or more agents (some examples of which are discussed above), and targeted to the area of the body in which the suture is placed, allowing the delivered agent to effect the local tissue (e.g., injured tissue and/or tissue in which to promote fibrosis). In this way, one or more types of agents may be inserted into the body and targeted locally, avoiding interaction with other parts of the body and/or with other (healthy) tissue.

As will be understood by those skilled in the art, NPs may contain encapsulated, dispersed, absorbed, or conjugated drugs, supplements, and/or other types of agents. As will be understood by those skilled in the art, NPs may include colloidal drug delivery systems comprising particles with a size range from 10 to 1000 nm in diameter, but other alternatives (e.g., particles larger than 1000 nanometers in size) for delivery of agents may be desirable for some implementations.

In one example, NPs may be coated by a polymer, which releases the coated agent(s) by controlled diffusion, or erosion from the core across the polymeric membrane or particle matrix. Accordingly, the solubility and diffusivity of an agent in the polymer membrane determines the release rate of the agent.

NP size and surface characteristics may be configured to achieve both passive and active agent targeting. Site-specific targeting can be achieved by attaching targeting ligands, such as antibodies or aptamers, to the surface of NPs, or by using guidance in the form of magnetic NPs. NPs may also control and sustain release of an agent during transport to, or at, the site of localization, altering agent distribution and subsequent clearance of the agent in order to improve therapeutic efficacy and reduce side effects.

Various nanoforms have been attempted as agent delivery systems, varying from biological substances, such as albumin, gelatin and phospholipids for liposomes, to chemical substances, such as various polymers and solid metal-containing NPs. In one embodiment, a suture may comprise a polymer-drug conjugate, which may have a high size variation normally not considered as an NP. As noted above, nanodelivery systems may be designed to have agents absorbed or conjugated onto the particle surface, encapsulated inside the polymer/lipid or dissolved within a particle matrix.

According to some embodiments, a nanoparticle, or other type of targeted agent delivery system, may comprise one or more types of agents. In one example, a nanoparticle (e.g., attached to a suture) may comprise two or more types of agents, such as a first agent that promotes a fibrotic effect in tissue and a second agent that promotes a regenerative effect in tissue (e.g., to improve a rate of healing of a wound).

In accordance with some embodiments, a suture (referred to in this disclosure as a “nano suture”) comprises at least one targeted agent delivery system comprising at least one nanoparticle. In one example, a nano suture comprises a nanoparticle comprising an agent promoting a fibrotic effect in tissue. In another example, a nano suture comprises a nanoparticle comprising an agent promoting regeneration in tissue (e.g., to increase the rate of healing of a wound in a uterine wall).

In accordance with some embodiments, a nano suture may comprise any number of nanoparticles. In some embodiments, a nano suture may comprise any number of different types of nanoparticles, which may differ with respect to structure, coating, and/or agent(s)).

In accordance with some embodiments, an extrusion, solvent, and/or molding process may be utilized to produce one or more types of sutures described in this disclosure. Plastic molding methods, for example, are known to those skilled in the art and include, but are not limited to, melt molding (e.g., injection molding, extrusion molding, and/or compression molding) and solution molding.

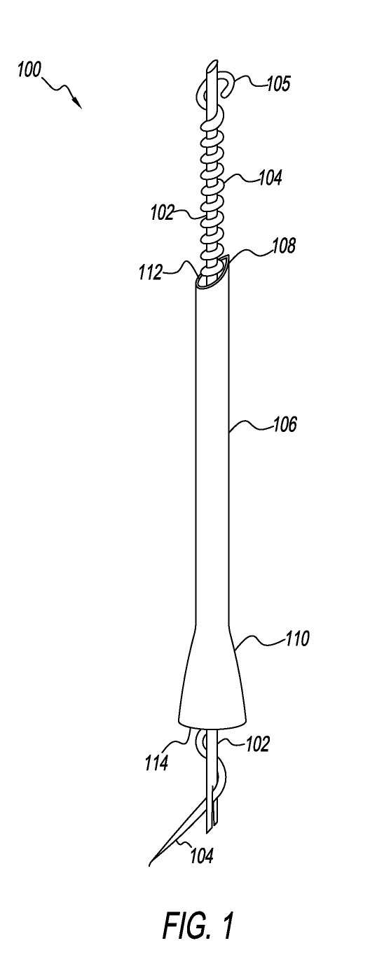

FIG. 1 depicts an example suture delivery device 100 for use with a coiled- or spring-type suture. The example suture delivery device 100 comprises a wire 102, around which a coiled- or spring-type suture 104 is wound at the insertion end of wire 102. The wire 102 and the suture 104 pass through the hollow interior of the sleeve needle 106, which has a rear opening 114 at rear portion 110 and a front opening 112 at front portion 108. Preferably the suture 104 comprises at least one fixating hook 105 at or proximate the insertion end of the suture 104.

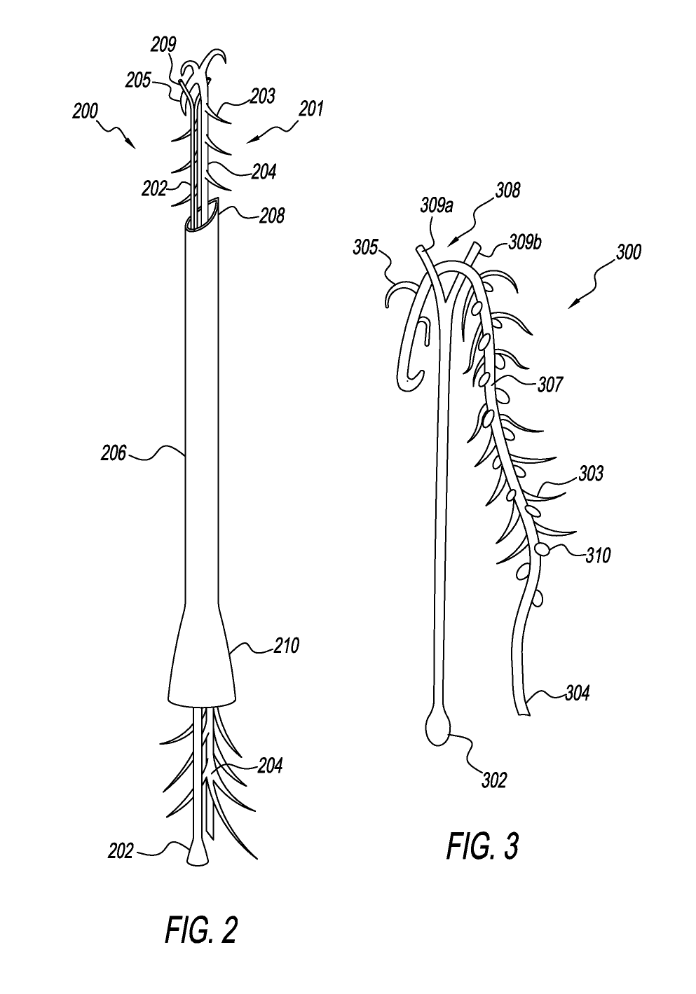

FIG. 2 depicts an example suture delivery device 200 for use with a non-spiral- or non-coiled-type suture 201. The example suture delivery device 200 comprises a wire 202 (also referred to as a “suture anchoring wire” or “anchoring wire”) having a suture retaining structure 209 (also referred to with respect to the embodiment depicted in FIG. 2 as a “fork”) at an insertion end of the wire 202. The suture retaining structure 209 preferably comprises a y-shaped fork (as depicted in FIG. 2) but alternatively may comprise two or more “tines” or other retaining structure suitable for catching, securing, or otherwise limiting the mobility of a suture 201 (e.g., as the wire 202 and the suture 201 are inserted into a patient's skin). As depicted in FIG. 2, the suture 201 preferably comprises at least one anchor 205 (e.g., for fixing the suture 201 in the body), and the anchor 205 may help the suture retaining structure 209 secure the suture 201 (e.g. during insertion) by preventing the suture 201 from slipping out of the suture retaining structure 209.

As depicted in FIG. 2, the suture 201 preferably comprises a suture body 204 from which extend a plurality of filaments 203. As described above, filaments 203 preferably are designed to be more flexible relative to the structures of other prior art suture devices that are designed to approximate tissue. The sleeve 206 preferably is hollow and comprises a front portion 208 and a rear portion 210, and the wire 202 and the suture 201 pass through the hollow interior of the sleeve 206.

FIG. 3 depicts an example system 300 comprising an example suture or thread 304 and an example wire 302. The thread 304 and the wire 302 may be used, in accordance with some embodiments, with a sleeve (e.g., like sleeve 206 of FIG. 2) to provide a suture delivery system (e.g., the suture delivery system 200 discussed with respect to FIG. 2). The thread 304 preferably comprises a thread body 307 and (i) a plurality of filaments 303 extending from the thread body 307 and/or (ii) one or more beads 310 (e.g., containing or comprising one or more drugs, vitamins, minerals, and/or other types of agents) attached to the thread body 307. In one embodiment, in addition to or instead of being connected to the thread body 307, a bead 310 may be attached to one or more of the filaments 303.

As shown in FIG. 3, in accordance with some embodiments, the thread 304 preferably comprises one or more anchors 305 for securing the thread 304 in living tissue.

The forked end 308 of the wire 302 preferably comprises tines or branches 309a and 309b, between which the thread 304 is passed and secured for the purposes of inserting the wire 302 and the thread 304 into a patient (e.g., for a lifting procedure and/or for a tissue approximation procedure).

Beads 310 are not necessarily represented to scale in FIG. 3. In accordance with some embodiments, as described in this disclosure, a bead 310 may represent a targeted delivery system comprising one or more nanoparticles, including one or more types of agents (e.g., an agent for producing a fibrotic effect and/or a second agent for accelerating healing or tissue regeneration).

According to some embodiments, a suture is provided that is comprised of an absorbable material and is also barbed (e.g., to facilitate fixing the suture in place subcutaneously during a rejuvenation or other cosmetic surgery procedure). In one embodiment, a suture comprises a thread comprised of an absorbable material (e.g., poliglecaprone 25, as used in Moncryl® absorbable suture by Ethicon, Inc.) characterized by delayed absorption (e.g., absorption within 8-12 weeks) and further configured to a plurality of barb-like structures. A delayed absorption of the suture may advantageously promote fibrosis for a longer period, while also advantageously resulting in eventual absorption of the suture.

The dimensions of a thread body, in accordance with some embodiments, may be of any size suitable for a desired surgical use. According to some embodiments, the diameter of the thread body of a suture may be configured specifically for use in a cosmetic surgery procedure (or one or more specific types of cosmetic surgery). In one embodiment, a diameter and/or length of the thread body may be configured for use in a face lift or other type of rejuvenative surgery procedure, which may require a diameter and/or length relatively smaller than for a suture used in cosmetic surgery involving other portions of the body (e.g., the abdomen).

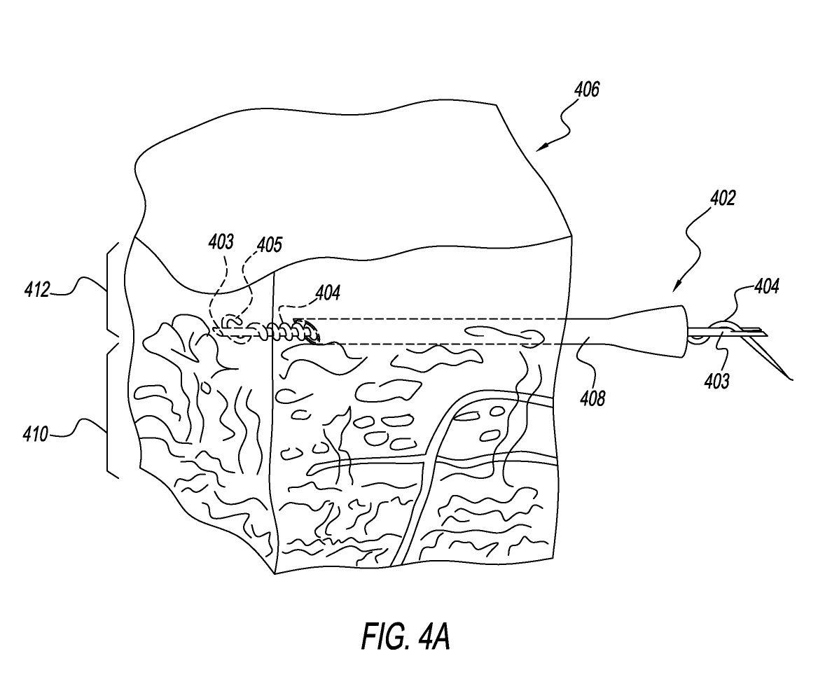

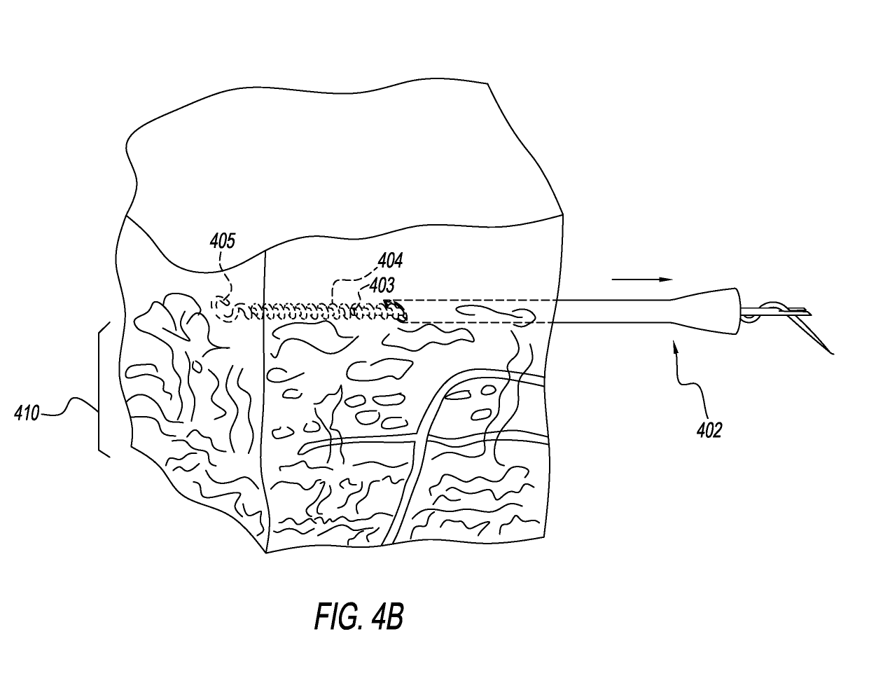

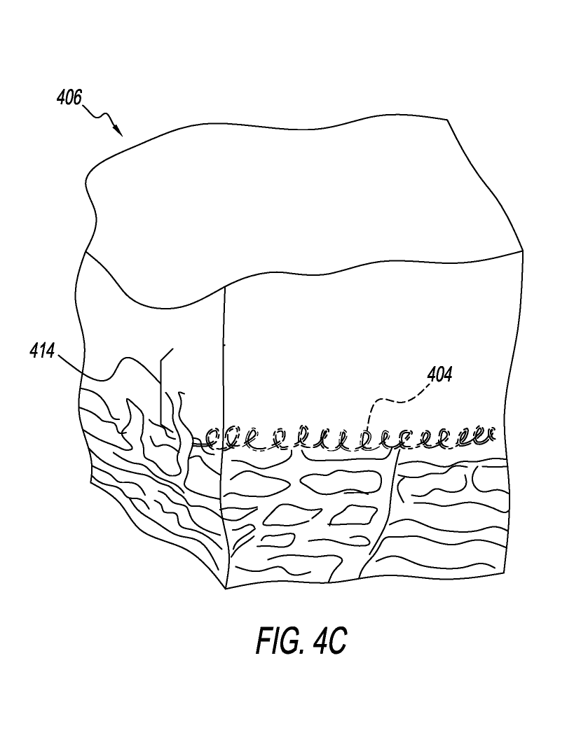

FIGS. 4A-4C depict an example sequence of an application of an embodiment of a suture delivery device. As depicted in FIG. 4A, a suture delivery device 402 is used to deliver a coiled suture 404 into tissue 406 (e.g., living tissue of a surgical patient in order). In some embodiments, the coiled suture delivery device 402 comprises a needle 408 (e.g., a hypodermic needle) that houses the coiled suture 404. In some examples, the needle 408 is driven into the tissue 406 (e.g. by a physician or other medical personnel), in the proximity of connective tissue portion 410.

In some embodiments, the location of delivery, including, for example, the depth that the needle 408 is driven into the tissue 406, may be determined based on the location of the connective tissue 410, and based on the nature of the task for which the sutures are being used (e.g., for facial rejuvenation). In one or more embodiments, the suture delivery device 402 may be placed into the tissue 406 to position the needle 408 such that it may deliver the inserting end of the coiled suture 404 into a position to secure the anchor 405 of the coiled suture 404 into the connective tissue 410. In some embodiments, the suture delivery device 402 may be inserted into a portion of the tissue 406 in which it is desirable to develop collagen or to produce some other fibrotic effect (e.g., in order to effect facial rejuvenation). In the example application of the suture delivery device 402 depicted in FIG. 4A, the suture delivery device 402 is being inserted into tissue portion or tissue layer 412, which contains no or relatively less connective tissue than connective tissue portion 410 does. In an alternative embodiment, the suture delivery device 402 may be inserted into a portion of the tissue 406 in order to approximate separated portions of the tissue (e.g., in order to close a wound or surgical incision).

As depicted in FIG. 4A, the suture delivery device 402 may further comprise a wire 403, around which the suture 404 is wound and passing through the needle 408, for assisting in placement of the suture 404, as described with respect to some embodiments in this disclosure.

FIG. 4B depicts a next step in the process of using the suture delivery device 402. Suture delivery device 402 is shown as being retracted out of tissue 406 (with the example wire 403), after the leading end of the coiled suture 404 has been inserted into the desired position. In some embodiments, the coiled suture 404 may be anchored into the connective tissue 410 (e.g., using anchor 405), or some other anchor point. As the suture delivery device 402 is pulled out of the tissue 406, the anchored coiled suture 404 remains in the tissue 406. In some embodiments, when the suture delivery device 402 is retracted from the tissue, the (preferably anchored) coiled suture 404 will freely slide through the hollow shaft (not shown) of the needle 408.

In some embodiments, the coiled suture 404 remaining in the tissue 406 after the suture delivery device 402 is retracted (as discussed with respect to FIG. 4B) may fully or partially dissolve and/or promote the growth of fibrotic tissue, as depicted in FIG. 4C at example fibrotic tissue portion 414.

The coiled suture 404 may, in some embodiments, contain and/or have adhered to it a chemical, biological, or structural component or other agent (not shown) that either speeds or slows the rate of dissolution, depending on the desired effect of the coiled suture 404 in the tissue. In one example, a nanoparticle may include a chemical structure for increasing the rate of dissolution. In one or more embodiments, the coiled suture 404 may remain whole, or may only partially dissolve. In some embodiments, as discussed in this disclosure, one or more chemical, biological, or structural moieties, including, for example, hormones or growth factors, may be included in the coiled suture 404 and/or in the suture delivery device 402, to enhance or inhibit, depending on the desired effect, the growth of fibrotic tissue and/or tissue regeneration.

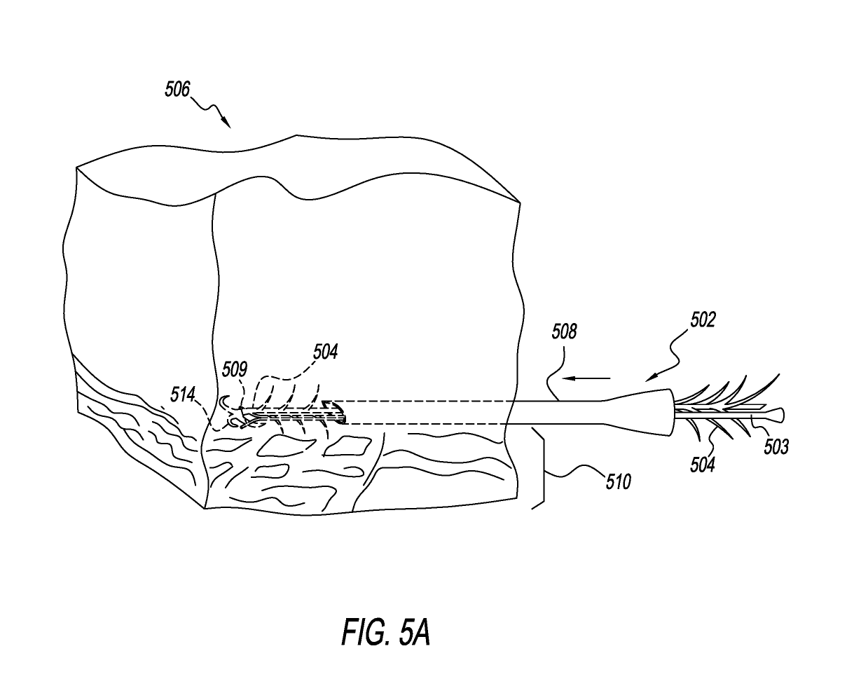

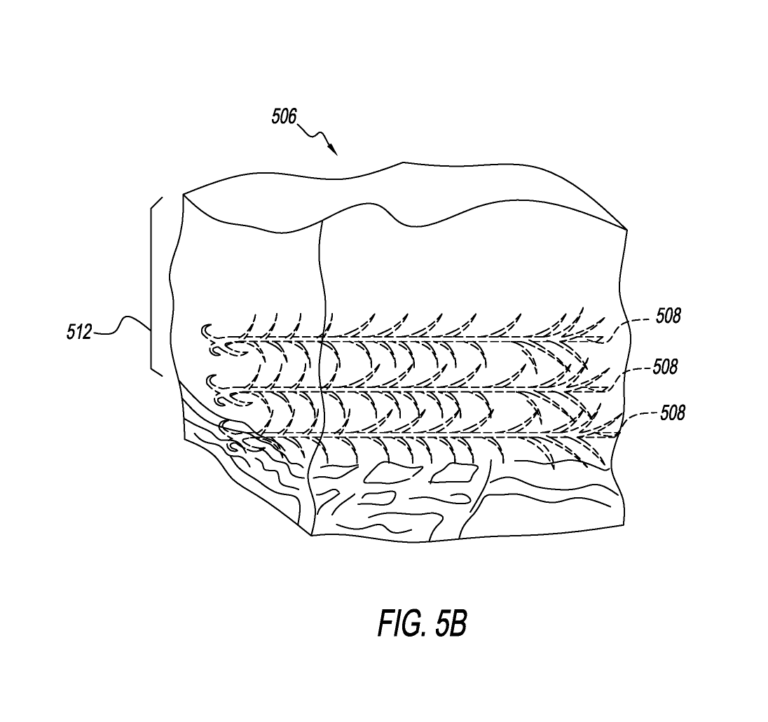

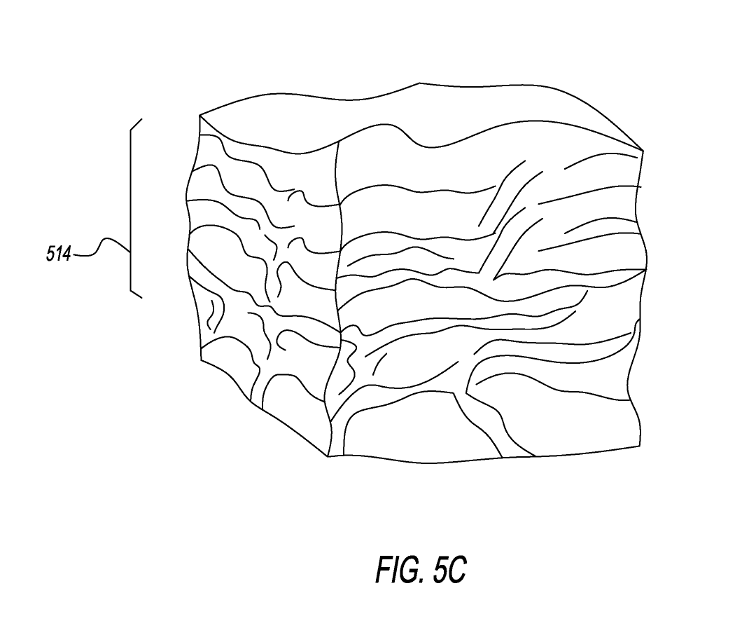

FIGS. 5A-5C depict an example sequence of an application of a suture delivery device 502 to deliver a barbed suture 504 into tissue 506. The suture delivery device 502 may comprise a needle 508 that is driven into the tissue 506 in the proximity of connective tissue portion 510. After being placed in the desired position (e.g., by a surgeon), the barbed suture 504 can be anchored into the surrounding tissue, for example, by retracting the suture delivery device 502 out of the tissue 506, allowing the barbs on the barbed suture 504 to retain the barbed suture 504 in the tissue 506 while the shaft of the hollow needle 508 is removed from around the barbed suture 504.

In one embodiment, as described in this disclosure, the suture delivery device 502 may further comprise a wire 503 with an insertion end 509 (e.g., a forked end for securing the barbed suture 504). In one or more embodiments, the barbed suture 504 may comprise at least one anchor 514 for securing the barbed suture 504 in the tissue.

In some instances it may be desirable to create more fibrous tissue to promote a more profound impact on surrounding tissues to create a desired effect. To achieve this effect, in some embodiments, it may be preferable to anchor multiple sutures (e.g., more than one barbed suture 504) into tissue to achieve a desired effect. For example, the tissue 506 depicted in FIG. 5B has three barbed sutures 508 that were delivered into place using the suture delivery device 502. In one or more embodiments, implanting multiple sutures may create a mesh-like effect that may promote the growth of fibrotic tissue, a better lifting or rejuvenation effect, and/or improve collagen synthesis, as depicted at fibrotic portion 514 of FIG. 5C. In some embodiments, a suture configured with multiple filaments extending from the central body of the suture (e.g., as depicted in FIG. 3) may create a mesh promoting collagen synthesis and/or a lifting effect when inserted in tissue, alone or in combination with one or more additional sutures. In some embodiments, as discussed in this disclosure, using one or more chemical, biological, and/or structural moieties, such as hormones or growth factors, may enhance fibrotic growth. In some embodiments, using multiple sutures may have an additive effect, creating more fibrotic growth, either linearly or exponentially, as a greater number of sutures are added.

According to some embodiments, a suture is provided. The suture preferably comprises a thread body comprising a distal end and a proximal end, and at least one filament attached to the thread body. In some embodiments, the thread body further comprises at least one nanoparticle affixed to one or more of the thread body and the at least one filament, wherein the distal end of the thread body comprises an anchoring structure configured to secure the thread body in living tissue.

In one embodiment of the suture described in the preceding paragraph, at least one of the at least one filament is adapted to promote the production of fibrotic tissue when placed within living tissue. In one embodiment of the suture described in the preceding paragraph, at least one of the at least one nanoparticle is adapted to promote the production of fibrotic tissue when placed within living tissue and/or at least one of the at least one nanoparticle is adapted to promote tissue regeneration when placed within living tissue.

In some embodiments, a nanoparticle (e.g., attached to a thread body of suture) comprises one or more of the following types of agents: a chemical agent, a biological agent, a hormonal agent, zinc, and/or plasma.

According to some embodiments, a filament of a suture may comprise one or more of the following: a filament mesh and/or a plurality of barbs.

### INTERPRETATION

Numerous embodiments are described in this patent application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognize that the disclosed invention may be practiced with various modifications and alterations, such as structural, logical, software, and/or electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is neither a literal description of all embodiments nor a listing of features that must be present in all embodiments.

Neither the Title (set forth at the beginning of the first page of this disclosure) nor the Abstract (set forth at the end of this disclosure) is to be taken as limiting in any way the scope of the disclosed invention(s).

Throughout the description and unless otherwise specified, the following terms may include and/or encompass the example meanings provided below. These terms and illustrative example meanings are provided to clarify the language selected to describe embodiments both in the specification and in the appended claims, and accordingly, are not intended to be limiting.

The terms “an embodiment”, “embodiment”, “embodiments”, “the embodiment”, “the embodiments”, “one or more embodiments”, “some embodiments”, “one embodiment” and the like mean “one or more (but not all) disclosed embodiments”, unless expressly specified otherwise.

The terms “the invention” and “the present invention” and the like mean “one or more embodiments of the present invention.”

A reference to “another embodiment” in describing an embodiment does not imply that the referenced embodiment is mutually exclusive with another embodiment (e.g., an embodiment described before the referenced embodiment), unless expressly specified otherwise.

The terms “including”, “comprising” and variations thereof mean “including but not limited to”, unless expressly specified otherwise.

The terms “a”, “an” and “the” mean “one or more”, unless expressly specified otherwise.

The term “plurality” means “two or more”, unless expressly specified otherwise.

The term “herein” means “in the present disclosure, including anything which may be incorporated by reference”, unless expressly specified otherwise.

The phrase “at least one of”, when such phrase modifies a plurality of things (such as an enumerated list of things) means any combination of one or more of those things, unless expressly specified otherwise. For example, the phrase at least one of a widget, a car and a wheel means either (i) a widget, (ii) a car, (iii) a wheel, (iv) a widget and a car, (v) a widget and a wheel, (vi) a car and a wheel, or (vii) a widget, a car and a wheel.

The phrase “based on” does not mean “based only on”, unless expressly specified otherwise. In other words, the phrase “based on” describes both “based only on” and “based at least on”.

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

Each process (whether called a method, algorithm or otherwise) inherently includes one or more steps, and therefore all references to a “step” or “steps” of a process have an inherent antecedent basis in the mere recitation of the term “process” or a like term. Accordingly, any reference in a claim to a “step” or “steps” of a process has sufficient antecedent basis.

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

As used in this disclosure, a “user” may generally refer to any individual and/or entity that operates a device.

A description of an embodiment with several components or features does not imply that any particular one of such components and/or features is required. On the contrary, a variety of optional components are described to illustrate the wide variety of possible embodiments of the present invention(s). Unless otherwise specified explicitly, no component and/or feature is essential or required.

Further, although process steps, algorithms or the like may be described or depicted in a sequential order, such processes may be configured to work in one or more different orders. In other words, any sequence or order of steps that may be explicitly described or depicted does not necessarily indicate a requirement that the steps be performed in that order. The steps of processes described in this disclosure may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications, does not imply that the illustrated process or any of its steps is necessary to the invention, and does not imply that the illustrated process is preferred.

While the present invention has been illustrated by a description of various embodiments and while these embodiments have been described in considerable detail, it is not the intention to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the general inventive concept.

The present disclosure provides, to one of ordinary skill in the art, an enabling description of several embodiments and/or inventions. Some of these embodiments and/or inventions may not be claimed in the present application, but may nevertheless be claimed in one or more continuing applications that claim the benefit of priority of the present application. Applicant reserves the right to file additional applications to pursue patents for subject matter that has been disclosed and enabled but not claimed in the present application.

What is claimed is:

1. A suture delivery device comprising:
a tubular sleeve with a hollow interior comprising a distal end and a proximal end, wherein the distal end comprises an opening and is adapted for insertion into tissue; and
a wire comprising a distal end and a proximal end, the wire housed within the hollow interior of the tubular sleeve,
wherein the distal end of the wire is adapted for insertion into tissue with the distal end of the tubular sleeve, and
wherein the wire is configured to hold a suture on insertion of the wire and the tubular sleeve into tissue and to leave the suture secured in the tissue on retraction of the wire and the tubular sleeve from the tissue.

2. The suture delivery device of claim 1, wherein the wire is fixed within the hollow interior of the tubular sleeve.

3. The suture delivery device of claim 1, wherein the wire is configured to move longitudinally within the hollow interior of the tubular sleeve.

4. The suture delivery device of claim 1, wherein the wire is configured to move freely within the hollow interior of the tubular sleeve.

5. The suture delivery device of claim 1, wherein the proximal end of the tubular sleeve comprises an opening.

6. The suture delivery device of claim 5, wherein a portion of the proximal end of the wire protrudes from the open proximal end of the tubular sleeve during insertion in tissue.

7. The suture delivery device of claim 1, wherein a portion of the distal end of the wire protrudes from the opening of the distal end of the tubular sleeve during insertion in tissue.

8. The suture delivery device of claim 1, wherein the suture is configured to dissolve after being placed in the tissue.

9. The suture delivery device of claim 1, wherein the suture is coiled around the wire.

10. The suture delivery device of claim 1, wherein the wire comprises a forked structure on the distal end of the wire to hold the suture.

11. The suture delivery device of claim 10, wherein the forked structure of the wire is configured to abut an anchor on a suture to hold the suture on insertion of the suture in tissue.

12. A suture comprising:
a thread body comprising a distal end and a proximal end;
the thread body comprising at least one element adapted to promote the production of fibrotic tissue when placed within living tissue; and
wherein the distal end of the thread body comprises an anchoring structure configured to secure the thread body in living tissue.

13. The suture of claim 12, wherein the anchoring structure is further configured to abut a holding structure of a suture delivery device on insertion of the thread body with the suture delivery device into tissue.

14. The suture of claim 12, wherein the at least one element is slanted toward the proximal end of the thread body.

15. The suture of claim 12, wherein the at least one element adapted to promote the production of fibrotic tissue comprises a nanoparticle.

16. The suture of claim 12, wherein the at least one element adapted to promote the production of fibrotic tissue is affixed to the thread body in a nanoparticle application process.

17. The suture of claim 12, wherein the at least one element adapted to promote the production of fibrotic tissue comprises a chemical agent.

18. The suture of claim 12, wherein the at least one element adapted to promote the production of fibrotic tissue comprises a biological agent.

19. The suture of claim 12, wherein the at least one element adapted to promote the production of fibrotic tissue comprises a hormonal agent.

20. The suture of claim 12, wherein the at least one element adapted to promote the production of fibrotic tissue comprises at least one of:
a plurality of flexible filaments,
a filament mesh,
a plurality of barbs,
a plurality of beads.
